(19) United States
(12) Patent Application Publication
(10) Pub. No.: US 2008/0119894 A1
(43) Pub. Date: May 22, 2008

(54) Osteosynthesis Plate, E.G. A Plate For The Head Of The Radius Or Humerus, With A Plurality Of Bore Holes For Receiving Bone Screws

(75) Inventors: Arnd Ehrhardt, Dornfeld (DE); Ulrich Finger, Saalfeld (DE); Frank Orschler, Konigsee (DE)

Correspondence Address: BODNER & O'ROURKE, LLP, 425 BROADHOLLOW ROAD, SUITE 108, MELVILLE, NY, 11747, US

(73) Assignee: Konigsee Implantate und instrumente zur Osteosynthese GmbH, Konigsee-Aschau (DE)

(21) Appl. No.: 11/794,439

(22) PCT Filed: Dec. 14, 2005
(86) PCT No.: PCT/EP05/13456

§ 371 Date: Nov. 21, 2007

(30) Foreign Application Priority Data
Dec. 30, 2004 (DE) 10 2004 063 595.1
Feb. 2, 2005 (DE) 10 2005 004 841.2

(51) Int. Cl. A61B 17/58

(52) U.S. Cl. 606/280; 606/60

(57) ABSTRACT

The invention relates to an osteosynthesis plate, particularly a plate for the head of the radius or humerus, provided with a plurality of bore holes for receiving bone screws, wherein at least some of the bore holes are provided with an inner thread and/or at least one continuous longitudinal bore hole is arranged in the plate, also having a conical shape extending over the longitudinal and/or transversal axis thereof. According to the invention, at least some of the bore holes also have a conical hollow space for receiving bone screws with both a head thread and a spherical head in the area near the bone. The thread section and hollow area section are disposed concentrically above or inside each other. The thread section can be embodied in such a way that it forms a conical thread.

The invention relates to an osteosynthesis plate, e.g. a plate for the head of the radius or humerus, with a plurality of bore holes for receiving bone screws, wherein at least some of the bore holes are provided with an inner thread and/or at least one continuous longitudinal hole is arranged in the plate, which may also have a conical shape extending over the longitudinal and/or transversal axis thereof, according to the preamble of patent claim 1.

An osteosynthesis plate, specifically an angularly stable radius plate for operatively treating bone fractures is already known from the German utility model DE 203 09 361. This plate comprises bore holes for receiving bone screws, wherein at least some of the bore holes are provided with an inner thread. Moreover, at least two of the bore holes are formed to partially engage and overlap each other. These overlapping bore holes each have a conical inner thread, wherein the engaging bore holes are arranged in the longitudinal and/or transverse direction of the plate or on a predefined radius.

With such an osteosynthesis plate it is possible to obtain substantially greater degrees of freedom in placing it on the bone, wherein the danger of bone cracks when driving the bone screws in is largely reduced.

An osteosynthetic bone plate comprising several round and/or longitudinal holes for receiving bone screws, which are arranged in the longitudinal direction, is known from DE 43 41 980 A1

The holes arranged in this prior bone plate have a conical thread, wherein the screw head of the bone screws likewise has a conical thread, but an external one. By providing the holes in the bone plate and the screw head of the bone screw with a conical thread it is achieved that the screw head gets jammed in the bone plate upon tightening it, so that the bone screws are thus firmly connected to the bone plate. According to this prior solution it is an advantage that the secure connection of the bone fragments is not achieved by pressing the bone plate against the bone surface, but only by the seat of the bone screws in the bone fragments, so that the bone skin is not affected additionally and the healing process can thus be accelerated.

At least one hole of the plate known from DE 43 41 980 A1 has a longitudinal shape which extends parallel to the longitudinal axis of the plate, wherein the radii at the ends have different dimensions.

The radius facing away from the bone fracture is smaller than the radius facing the bone fracture. Further, the longitudinal hole is formed conically and is provided with a peripheral inner thread. The conical form of this hole, again, corresponds to that of the screw head of a corresponding bone screw. The tightening of the bone screw then initiates a movement lengthwise of the bone plate and, thus, a compression of the bone fragments. According to DE 43 41 980 A1 a narrow placement of several screws, if necessary also smaller diameter ones, on a bone piece is not possible. If several holes each having a longitudinal form were adjacent, stability problems would occur due to a weakening of the plate material. This could, again, only be compensated by a greater thickness, which undesirably raises the entire structure, however, because of the greater plate thickness.

The osteosynthesis plate according to EP 0 468 192 A2 is provided with several bore holes for receiving bone screws, said bore holes connecting the upper side and the underside of the plate. According to the preferred solution thereof, the plate holes are formed conically and are preferably provided with an inner thread. The screws used for fixing the plate have a head that tapers off conically, preferably including a conical outer thread. The screws are screwed through the plate holes into the bones in a manner known per se. Upon completely screwing in the screw, the conical screw head braces in the conical bore of the plate, whereby this effect is supported by the preferably incorporated threads. This aforementioned bracing is essential if the screw is to be inserted only unicortally and the plate does not rest on the surface of the bone. The conical screw shape and a corresponding conical connection is advantageous because the threads become wedged with each other upon tightening them firmly. This wedging reduces the danger of an unintended loosening of the plate/screw connection in the event of corresponding cyclically recurring stresses.

With respect to the prior art, reference is additionally made to the screwed connection used in osteosynthesis according to DE 200 22 673 U1. The screw employed therein has a substantially conical section in the longitudinal direction. Moreover, a ring is provided the outer surface of which has a spherical or spherical segment shape and which is open or is separated at one point. In the implant, specifically in a tibia head plate, a recess in the form of a bore hole is provided for mounting the ring. Moreover, a bearing ring attachable in the bore hole is provided, with the inner surface of the bearing ring having a complementary form with respect to the outer surface of the ring, so that the ring mounted in the bearing ring can be moved in a ball-joint type manner. The ring is expandable by a conical section of the screw, so that the outer surface of the ring can be pressed against the inner surface of the bearing ring, which permits that the angular position of the screw is fixed with respect to the implant. The inserted screw is preferably embodied as a tibia head screw having a spongiosa thread, as a support screw having a corticalis thread or as a condyle screw having a conical thread at the head. In addition, the condyle screw may be screwed together with a nut so as to form a compression screw.

Finally, EP 1 143 867 B1 shows a fixation system for bones for use in osteosynthesis, wherein bone screws are connected to bone ends and the connection carrier, i.e. the plate, bridges over the fracture. To allow a mutual alignment under different angles and to fix the bone screw at a specific angle with respect to the connection carrier, the means for fixing comprise a thread connection below the seat surfaces of the bone screw and connection carrier, which is formed by rotating in the bone screw at a certain angle by a preformed thread below at least one of the seat surfaces by way of deformation. The thread connection is formed by a preformed thread below the one seat surface and one at least partly circumferential projection below the other seat surface, this projection being deformable by rotating in the preformed thread. The seat surfaces may be spherical or conical. One seat surface according to EP 1 143 867 B1 is formed on the lower side of a screw head of the bone screw. In the fixation system for bones according to EP 1 143 867 B1, therefore, a deformation between the materials of different hardness always takes place with a large amount of force, namely to the extent that not only a force in terms of a friction fit is provided, but also a material fit (friction welding).

In the solutions described above it is basically necessary to employ, in dependence on the embodiment of the fixing bore holes or fixing holes with or without a thread or deformable section correspondingly complementary screws, which is a contradiction to the idea of a possibly large standardization with a small stockpiling.

Furthermore, the space for providing bore holes or holes in an osteosynthesis plate is basically limited. The number, but also the size of the holes cannot be chosen optionally because the stability of the plate is otherwise inadmissibly weakened. Therefore, in order to be prepared for all cases of application, a plurality of plates having different hole configurations would have to be kept in stock. This, too, causes an extensive stock-keeping with higher costs.

Based on the foregoing it is therefore the object of the invention to provide an advanced osteosynthesis plate, specifically a plate for the head of the radius or humerus, with a plurality of bore holes for receiving bone screws, wherein at least some of the bore holes are provided with an inner thread. The osteosynthesis plate to be provided is to permit, at nearly any location, an angularly stable screw connection by means of head attachment screws as well as a fixation by means of common spherical screws. That is, it is to be usable in a multifunctional manner.

The solution to the object of the invention is achieved with an osteosynthesis plate according to the combination of the features defined in patent claim 1, wherein the dependent claims represent at least useful embodiments and advancements.

Accordingly, the basic idea of the invention resides in that the profiles of holes having a conical thread and a common spherical cross-section are disposed above each other so as to form a combined hole for implants, said hole being suited for providing a screw connection optionally in an angularly stable manner by means of head attachment screws, but also by means of spherical head screws. For this purpose, the combined hole is formed as an angularly stable hole in the lower plate area near the bone. In the upper area of the plate the hole has a shape to receive common spherical head screws.

If the at least one continuous longitudinal hole is formed as a compression hole and has a peripheral conical thread, with a spherical countersink being incorporated in a partial section in the area of the longitudinal hole away from the bone, it is possible to optionally use this area for screwing the corresponding bone fragments together with or without compression.

According to the invention, at least some of the bore holes are suited to receive bone screws with both a head thread and a spherical head, and comprise a thread shape in the area near the bone and a spherical countersink in the area away from the bone.

The thread section and the countersink section are disposed concentrically above or inside each other.

The thread section is preferably embodied to form a conical thread.

The depth of the countersink for obtaining the spherical shape is chosen to be equal to or smaller than the thickness of the respective plate.

The combined holes or bore holes having a spherical countersink and a thread section are preferably formed in the shaft region of the plate.

At least two bore holes having a spherical countersink and a thread section may be embodied to engage each other or laterally overlap each other.

Also, it is possible that the diameter of the engaging, overlapping bore holes adopts different values.

The countersink for obtaining the spherical shape is embodied in such a way that the spherical screw head coming to rest therein is nearly flush with the upper surface or upper side of the plate.

The dimension of countersinking the sphere is chosen such that the distance of the central point of the sphere from the side of the plate in the area near the bone in the lowest countersinking position is in the range between 0.9 times and 1.2 times the sphere radius, preferably 1.0 to 1.1 times.

The dimension of countersinking the sphere in the highest position is, again, chosen such that the distance of the central point of the sphere from the side of the plate in the area near the bone is greater than in the lowest position, but smaller than the sum of the plate strength or plate thickness and the sphere radius, preferably 1.1 to 1.3 times the radius, however.

Below, the invention shall be explained in more detail by means of exemplary embodiments with the aid of the figures. In the drawings:

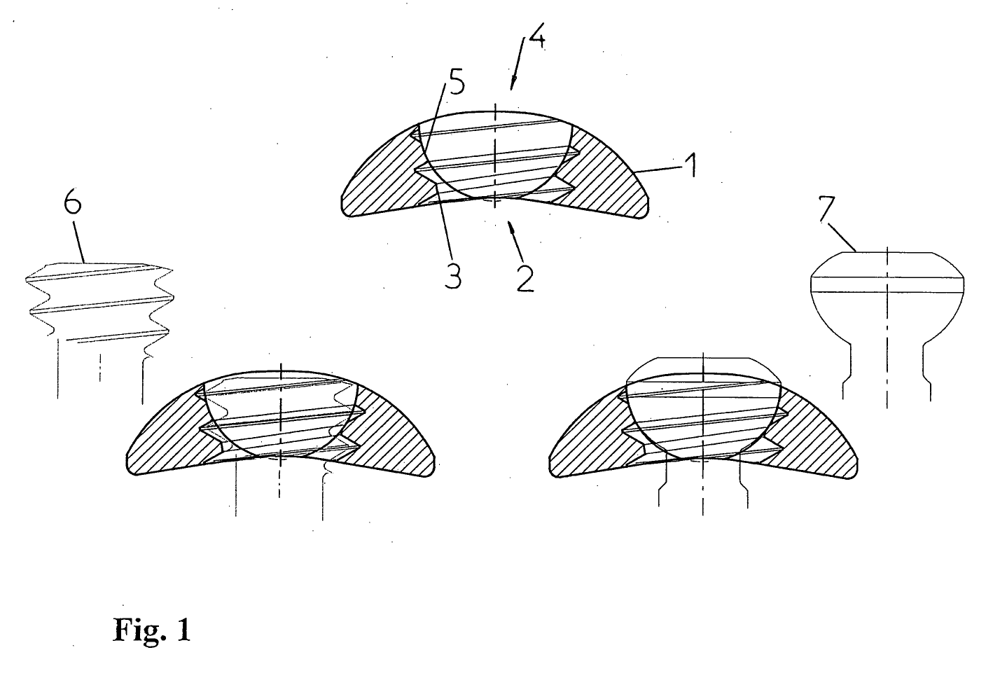

FIG. 1 shows a cross-sectional view of a large-fragment plate comprising a combined hole, wherein the left part of the picture shows a screw with a conical head located in the bore hole, while it is illustrated in the right part of the picture how a screw with a common spherical head is located in the corresponding bore hole in a screwed-in position;

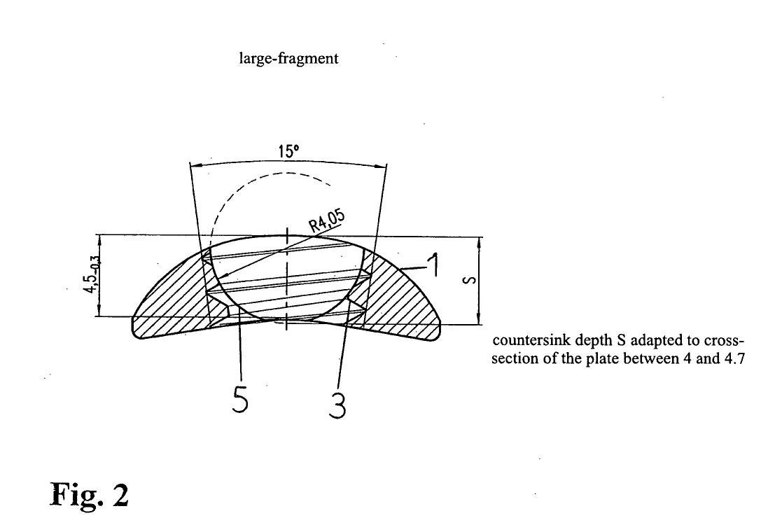

FIG. 2 shows a detailed view of a cross-section through a large-fragment plate with a combined hole embodied therein;

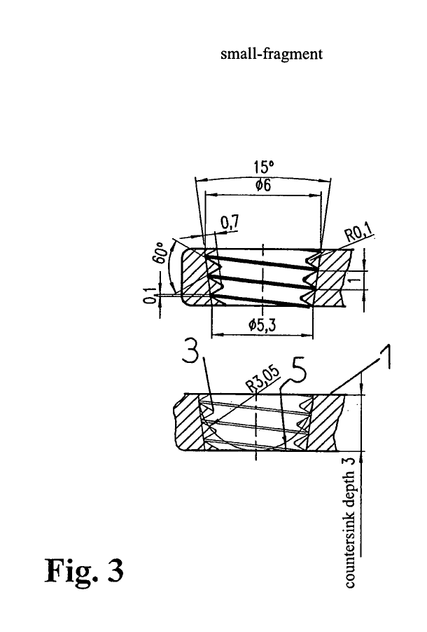

FIG. 3 shows a view of a small-fragment plate with a spherical countersink for forming the combined hole recognizable in the lower part of the picture;

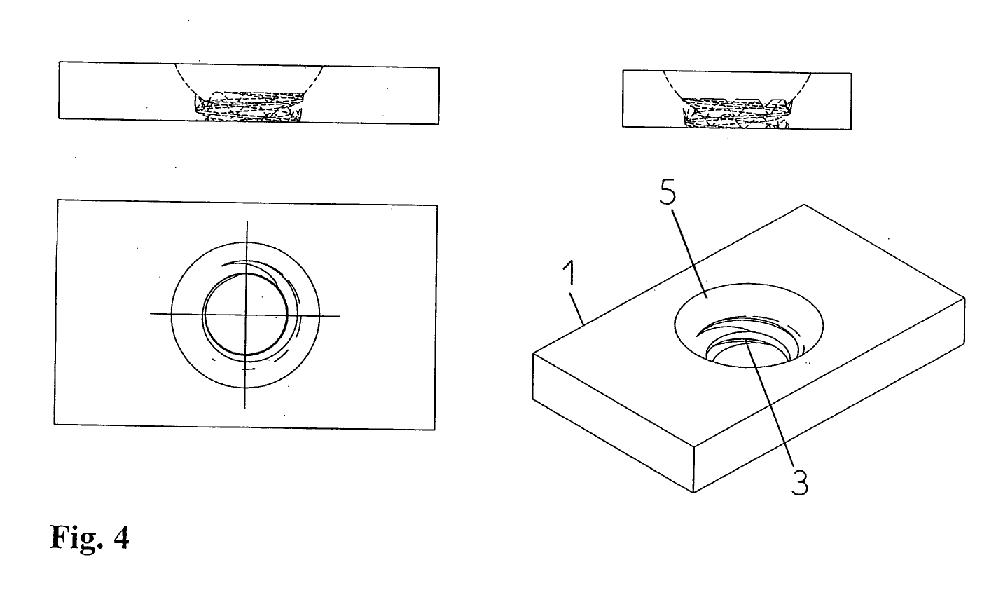

FIG. 4 shows a top view, a sectional view and a three-dimensional view of a combined hole for angularly stable screws or for screws having a spherical head bottom side; and

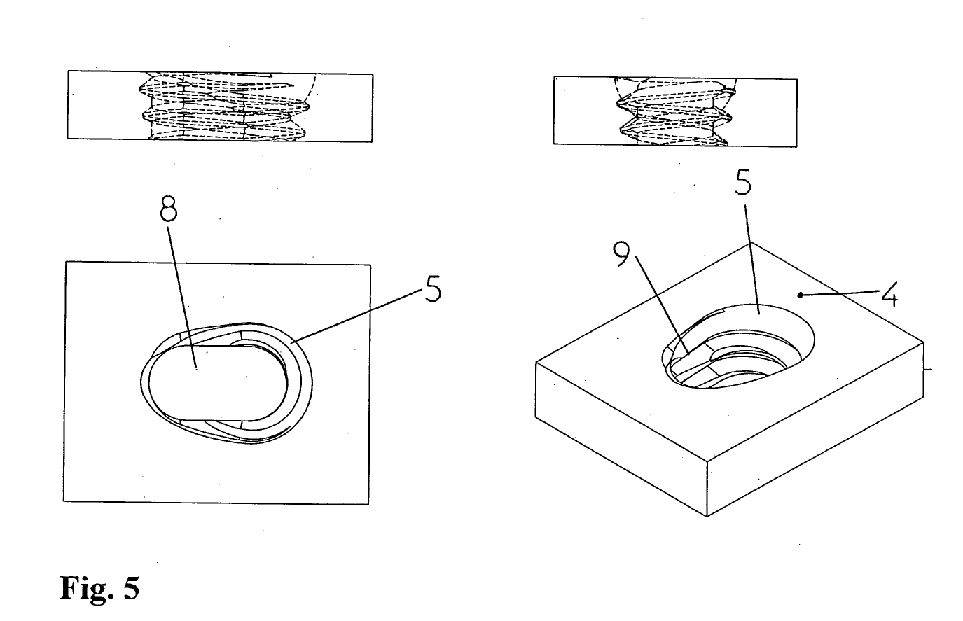

FIG. 5 shows an illustration similar to that of FIG. 4, but including a longitudinal hole embodied as an angularly stable dynamic compression hole for both angularly stable screws and screws having a spherical head bottom side.

According to FIG. 1, an osteosynthesis plate 1 has a crescent-shaped cross-section.

The plate comprises, for example, in a (non-illustrated) shaft part several bore holes located on the longitudinal axis. According to the invention the bore holes are suited to receive bone screws with both a head thread and a spherical head.

To this end, the bore hole has a thread shape in the area near the bone 2, preferably embodied as a conical thread 3.

In the area away from the bone 4, i.e. on the upper side of the plate, a spherical countersink is formed, wherein the thread section and the countersink section are disposed concentrically above each other and partially overlap with each other to form a sphere, as is illustrated in FIG. 1.

Therefore, the so embodied combined hole is suited to receive both a screw with a threaded head 6 and a screw with a spherical head 7 without requiring any modifications to the plate, a subsequent treatment or the use of a special screw.

The depth of the countersink for obtaining the spherical shape is chosen to be equal to or slightly smaller than the plate thickness. An exemplary dimensioning for a large-fragment plate comprising a combined hole according to the invention is shown in FIG. 2.

Of course, the combined hole can analogously also be realized for small-fragment plates, as is illustrated in FIG. 3.

According to the illustration in FIG. 4 showing a combined hole for angularly stable screws or screws having a spherical head bottom side, the respective threaded portion is here incorporated in the lower half of plate 1. In this embodiment, the contact surface of the spherical countersink relative to the complementary surface of a screw having a spherical head is enlarged.

In one embodiment of a continuous longitudinal hole according to FIG. 5 said hole is embodied as a compression hole and comprises a peripheral conical thread 9. This plate, which is provided with a special hole, likewise has a spherical countersink 5 incorporated therein in a partial section in an area away from the bone 4. This plate, too, allows the use of both angularly stable screws having a conical head and common screws having a spherical head, namely in such a manner that the desired compression of bone fragments with respect to each other in the longitudinal direction is not limited. The diameter of screw heads for use in the combined holes according to the invention may be within the range between 1.0 mm and 12.0 mm. The cone angle of the corresponding hole is within the range of 5° to 20°, preferably 12° to 18°. The thread pitch, i.e. the distance from thread to thread is in the range of 0.3 mm to 2.5 mm, preferably 0.5 mm to 2.0 mm.

According to the embodiment of FIG. 5, the compression distances can be realized within the range of 0.5 mm to 6.0 mm, preferably 1.0 mm to 5.0 mm.

The countersink for the use of screws having a spherical head, i.e. the spherical segment, is realized in such a way that screw heads having a diameter from 1.0 mm to 12 mm, preferably in the range from 3.0 mm to 9 mm, can be used. In the lowest position, the distance of the countersink in the upper side of the plate from the central point of the sphere to the lower side of the plate in the area near the bone is greater than 0.9 times the radius of the sphere and smaller than 1.2 times the radius, preferably 1.0 to 1.1 times.

The highest position is chosen such that a distance of the central point of the sphere from the lower side of the plate in the area near the bone is greater than in the lowest position, wherein the distance is smaller than the sum of the plate strength and the radius, preferably 1.1 to 1.3 times the radius.

Summarizing, if holes and bore holes in osteosynthesis plates are embodied in accordance with the invention, any standard screw with both a spherical and a conical head, i.e. threaded head, can be used.

In comparison with solutions in which adjacent bore holes are engaging each other, with one bore hole having a conical thread and the opposite one having a countersink, the invention does not involve any weakening of the plate, and there remains no undesired place in the plate as a result of the unused section or unused bore hole, respectively.

### LIST OF REFERENCE NUMERALS

1 osteosynthesis plate
2 area near the bone
3 conical thread
4 area away from the bone
5 spherical countersink
6 screw having a threaded head
7 screw having a spherical head
8 continuous longitudinal hole
9 peripheral conical thread

1. An osteosynthesis plate, e.g. a plate for the head of the radius or humerus, provided with a plurality of bore holes for receiving bone screws, wherein at least one of the bore holes is provided with an inner thread and/or at least one continuous longitudinal bore hole is arranged in the plate, which may also have a conical shape extending over the longitudinal and/or transversal axis thereof,
characterized in that
at least one bore hole for receiving bone screws having both a head thread and a spherical head has a thread shape in the area near the bone and a spherical countersink in the area away from the bone, wherein the thread section and the countersink section are disposed concentrically above or inside each other.

2. The osteosynthesis plate according to claim 1,
characterized in that
the thread section is embodied as a conical thread.

3. The osteosynthesis plate according to claim 1 or 2,
characterized in that
the depth of the countersink for obtaining the spherical shape is chosen to be equal to or smaller than the plate thickness.

4. The osteosynthesis plate according to claim 1,
characterized in that
at least one continuous longitudinal hole is embodied as a compression hole and has a peripheral conical thread, wherein a spherical countersink is incorporated in a partial section in the area of the longitudinal hole away from the bone.

5. The osteosynthesis plate according to claim 1,
characterized in that
the combined holes or bore holes comprising a spherical countersink and a thread section are preferably formed in the shaft region of the plate.

6. The osteosynthesis plate according to claim 1,
characterized in that
at least two bore holes having a spherical countersink and a thread section are embodied to engage and laterally overlap each other.

7. The osteosynthesis plate according to claim 6,
characterized in that
the diameter of the engaging, overlapping bore holes adopts different values, specifically that the diameter of one of the bore holes is substantially smaller than that of the adjacent bore hole.

8. The osteosynthesis plate according to claim 1,
characterized in that
the countersink has such a depth that the spherical screw head coming to rest therein is nearly flush with the upper surface of the plate.

9. The osteosynthesis plate according to claim 1,
characterized in that
the dimension of countersinking the sphere is chosen such that the distance of the central point of the sphere from the side of the plate in the area near the bone in the lowest countersinking position is in the range between 0.9 times and 1.2 times the sphere radius, preferably 1.0 to 1.1 times.

10. The osteosynthesis plate according to claim 9,
characterized in that
the dimension of countersinking the sphere in the highest position is chosen such that the distance of the central point of the sphere from the side of the plate in the area near the bone is greater than in the lowest position, but smaller than the sum of the plate strength or plate thickness and the sphere radius, preferably at 1.1 to 1.3 times the radius, however.

11. The osteosynthesis plate according to claim 4,
characterized in that
the longitudinal hole type compression hole is formed such that a peripheral conical thread is overlapped by a countersink in the area of the longitudinal hole away from the bone, which is incorporated by translationally countersinking the sphere with a simultaneous linear change of the countersinking depth.

12. The osteosynthesis plate according to claim 11,
characterized in that
the depth of the spherical countersink in the highest position is smaller than in the lowest position, wherein the highest position and the lowest position are each smaller than the sum of the plate strength and the sphere radius.

13. The osteosynthesis plate according to claim 11,
characterized in that
the compression holes are preferably formed in the shaft region of the plate.

14. The osteosynthesis plate according to claim 2,
characterized in that
the depth of the countersink for obtaining the spherical shape is chosen to be equal to or smaller than the plate thickness.

15. The osteosynthesis plate according to claim 2,
characterized in that
the combined holes or bore holes comprising a spherical countersink and a thread section are preferably formed in the shaft region of the plate.

16. The osteosynthesis plate according to claim 3,
characterized in that
the combined holes or bore holes comprising a spherical countersink and a thread section are preferably formed in the shaft region of the plate.

17. The osteosynthesis plate according to claim 4,
characterized in that
the combined holes or bore holes comprising a spherical countersink and a thread section are preferably formed in the shaft region of the plate.

18. The osteosynthesis plate according to claim 2,
characterized in that
at least two bore holes having a spherical countersink and a thread section are embodied to engage and laterally overlap each other.

19. The osteosynthesis plate according to claim 3,
characterized in that
at least two bore holes having a spherical countersink and a thread section are embodied to engage and laterally overlap each other.

20. The osteosynthesis plate according to claim 12,
characterized in that
the compression holes are preferably formed in the shaft region of the plate.
